(19) United States
(12) Patent Application Publication
(10) Pub. No.: US 2023/0151406 A1
(43) Pub. Date: May 18, 2023

(54) NUCLEIC ACID SEQUENCE DETECTION BY ENDONUCLEASE DIGESTION AND MASS SPECTROMETRY

(71) Applicant: Chan Zuckerberg Biohub, Inc., San Francisco, CA (US)

(72) Inventor: Emily D. Crawford, San Francisco, CA (US)

(21) Appl. No.: 17/797,680

(22) PCT Filed: Feb. 16, 2021
(86) PCT No.: PCT/US21/18244

§ 371 Date: Aug. 4, 2022

Related U.S. Application Data
(60) Provisional application No. 62/978,666, filed on Feb. 19, 2020.

(51) Int. Cl. C12Q 1/6806

(52) U.S. Cl. CPC C12Q 1/6806

(57) ABSTRACT

A method of nucleic acid analysis is described, the method including the steps of (a) providing a sample comprising a plurality of end-blocked polynucleotides derived from a biological source; (b) digesting at least some of the end-blocked polynucleotides with a nucleic acid-directed endonuclease that targets a sequence of interest to produce polynucleotide fragments that comprise the sequence of interest and a ligatable end generated by endonuclease cleavage; (c) ligating a moiety to the ligatable end to produce a moiety-target polynucleotide construct; and (d) detecting the moiety-target polynucleotide construct or a transcription or translation produce produced from the moiety-target polynucleotide construct using mass spectrometry. The moiety may be an adaptor sequence with a promoter for RNA polymerase. The moiety may be a chemical moiety that is highly amenable to flight and detection in a mass spectrometer.

### REFERENCE TO RELATED APPLICATION

This international (PCT) patent application claims the priority benefit of U.S. provisional patent application No. 62/978,666, filed Feb. 19, 2020. The propority application is hereby incorporated herein in its entirety for all purposes.

### FIELD

The present invention relates to the field of polynucleotide composition analysis, particularly to methods for detecting nucleic acid sequences in a polynucleotide composition.

### BACKGROUND

Massively parallel sequencing is used in a clinical setting with increasing frequency, for example to consider a patient's genome sequence or the genome sequence of a viral or bacterial pathogen when developing treatment plans. However, certain factors limit the broad application of this aspect of precision medicine. One limitation is the difficulty of rapidly identifying large numbers of nucleic acid sequences present at low abundance in clinical samples. For example, given a clinical sample from patient it can be difficult to selectively amplify and analyze a pathogen sequence from the background of human or other microbial sequences present in a biological sample.

Other limitations include the accessibility and speed of gene sequencing in a clinical setting. Many clinical laboratories do not have ready access gene sequencing technology and, even when available, conventional gene sequencing technology can take a day or longer to complete analysis of a polynucleotide composition.

Thus, a need exists for a method for analyzing a polynucleotides in samples, particularly low abundance polynucleotides in clinical samples.

### BRIEF SUMMARY OF THE DISCLOSURE

The invention relates to methods for genetic analysis such as an assay for the presence or absence of a nucleic acid sequence of interest in a biological sample. In one aspect, the method of genetic analysis involves (a) providing a complex mixture comprising a plurality of end-blocked polynucleotides; (b) digesting at least some of the end-blocked polynucleotides with a nucleic acid-directed endonuclease that cleaves a target polynucleotide comprising a sequence of interest to produce, if the sequence of interest is present in the complex mixture, a polynucleotide that includes the sequence of interest and at least one ligatable end generated by endonuclease cleavage; (c) ligating a moiety to a ligatable end of the polynucleotide under conditions in which the moiety cannot be ligated to a polynucleotide that lacks a ligatable end to produce a moiety-target polynucleotide construct; (d) detecting, using mass spectrometry, the moiety-target polynucleotide construct or a transcription or translation product produced from the moiety-target polynucleotide; (e) correlating the detection of the moiety-target polynucleotide with the presence of the sequence of interest in the complex mixture.

In a related aspect, the method of genetic analysis involves (a) providing a complex mixture comprising a plurality of end-blocked polynucleotides; (b) digesting at least some of the end-blocked polynucleotides with a nucleic acid-directed endonuclease that cleaves a target polynucleotide comprising a sequence of interest to produce, if the sequence of interest is present in the complex mixture, a polynucleotide that includes the sequence of interest and at least one ligatable end generated by endonuclease cleavage; (c) ligating a moiety to a ligatable end of the polynucleotide under conditions in which the moiety cannot be ligated to a polynucleotide that lacks a ligatable end to produce a moiety-target polynucleotide construct, wherein the moiety is a nucleotide adaptor comprising an RNA promoter and the moiety-target polynucleotide construct is configured so that transcription initiated at the promoter transcribes at least a portion of the sequences of interest to produce RNA products; translating the RNA products to produce a polypeptide or mixture of polypeptides; (f) detecting, using mass spectrometry, the polypeptide or mixture of polypeptides; and (e) correlating the detection of the polypeptide or mixture of polypeptides with the presence of the sequence of interest in the complex mixture. In some cases, the in vitro transcription and translation are coupled.

In a related aspect, the method of genetic analysis involves (a) providing a complex mixture comprising a plurality of end-blocked polynucleotides; (b) digesting at least some of the end-blocked polynucleotides with a nucleic acid-directed endonuclease that cleaves a target polynucleotide comprising a sequence of interest to produce, if the sequence of interest is present in the complex mixture, a polynucleotide that includes the sequence of interest and at least one ligatable end generated by endonuclease cleavage; (c) ligating a moiety to a ligatable end of the polynucleotide under conditions in which the moiety cannot be ligated to a polynucleotide that lacks a ligatable end to produce a moiety-target polynucleotide construct, wherein the moiety is a nucleotide adaptor comprising an RNA promoter and the moiety-target polynucleotide construct is configured so that transcription initiated at the promoter transcribes at least a portion of the sequences of interest to produce RNA products; (e) detecting, using mass spectrometry, the RNA products; and (e) correlating the detection of the RNA products with the presence of the sequence of interest in the complex mixture.

In a related aspect, the method of genetic analysis involves (a) providing a complex mixture comprising a plurality of end-blocked polynucleotides; (b) digesting at least some of the end-blocked polynucleotides with a nucleic acid-directed endonuclease that cleaves a target polynucleotide comprising a sequence of interest to produce, if the sequence of interest is present in the complex mixture, a polynucleotide that includes the sequence of interest and at least one ligatable end generated by endonuclease cleavage; (c) ligating a moiety to a ligatable end of the polynucleotide under conditions in which the moiety cannot be ligated to a polynucleotide that lacks a ligatable end to produce a moiety-target polynucleotide construct, wherein the moiety is a an adaptor linked to a mass label to produce adaptored polynucleotides linked to the mass label; (e) detecting, using mass spectrometry, the RNA adaptored polynucleotides linked to the mass label; and (e) correlating the detection of the adaptored polynucleotides linked to the mass label with the presence of the sequence of interest in the complex mixture.

In some cases the nucleic acid-directed endonuclease is a CRISPR-associated protein (Cas) and an associated guide RNA. In some cases the sequence of interest is a gene sequence of a strain of bacteria.

### BRIEF DESCRIPTION OF THE DRAWINGS

The invention is described in detail below with reference to the appended drawings.

FIGS. 1A-1C are flowcharts of exemplary methods of analysis in accordance with various embodiments of the present disclosure. FIG. 1A outlines the assay process when the mass analyte is a polypeptide. FIG. 1B outlines the assay process when the mass analyte is an RNA. FIG. 1C outlines the assay process when the mass analyte is DNA tagged with a flight moiety.

### DETAILED DESCRIPTION OF THE DISCLOSURE

Described herein are methods for analyzing compositions of polynucleotides by selective endonuclease digestion and subsequent analysis by mass spectrometry.

### 1. Protocol Overview

Using the method described herein, a low abundance nucleic acid sequence of interest is detected in a large background of other sequences. A composition comprising a complex mixture of polynucleotides is provided. The polynucleotides may be fragments of larger polynucleotides (such as fragments of chromosomal DNA). The fragments are treated to make the termini unligatable or “end-blocked.” At least some of the end-blocked polynucleotides are digested by one or more nucleic acid-directed endonuclease, also known as a programmable endonuclease, such as a guide RNA-Cas9 protein complex. A “programmable” endonuclease is a nucleic acid-protein complex that cleaves double stranded DNA in a sequence-specific fashion where the specificity is determined by the sequence of the nucleic acid component. The programmable endonuclease(s) in this example is designed to target and digest at sites (arrow heads) flanking sequences of interest. This produces nucleic acid fragments that comprise the sequence of interest and at least one ligatable end. Other end-blocked polynucleotides (such as host genomic DNA) do not include the sequence of interest and are not cleaved. Adapters are ligated to the ligatable ends. The adapters may contain regulatory elements that direct transcription of the adjacent sequences of interest and the RNA or polypeptides translated from the RNA are detected and characterized by mass spectrometry. In still other embodiments, a flight moiety is attached to the ligatable end(s) of a fragment.

Multiple approaches for detection of low abundance nucleic acid sequences of interest by endonuclease digestion and mass spectrometry are described in detail below. General methodology useful in the present invention is described in PCT Publication WO2018035062A1 and Quan et al., 2019, Nucleic Acids Research 47:14 e83, both of which are incorporated herein by reference for all purposes.

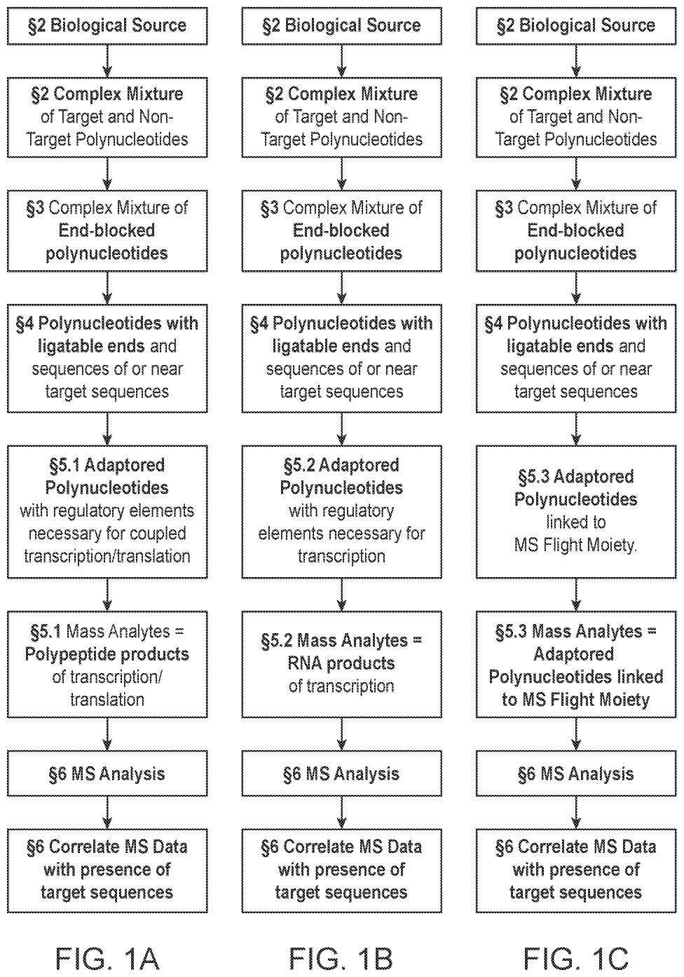

In a first approach of the present invention, oligonucleotide adaptors comprising regulatory elements for coupled in vitro transcription/translation are ligated to ligatable ends of at least some of the nucleic acid fragments containing sequences of interest. Addition of regulatory elements (e.g., promoters for RNA polymerase) to ligatable ends results in “adaptored fragments.” In this approach in vitro transcription and translation from the adaptored fragments is carried out to generate translation product(s) which may be referred to as “signature polypeptides” or a “signature polypeptide mixture.” The translation products are analyzed, e.g., using mass spectrometry. By programming the endonuclease to cleave at suitable positions, a signature polypeptide with a unique mass, or multiple signature polypeptides with a unique mass pattern will be produce when and only when the sequence of interest is present in the starting composition. See FIG. 1A and § 5.1 below.

In a second approach of the present invention, oligonucleotide adaptors comprising regulatory elements for in vitro transcription are ligated to ligatable ends of at least some nucleic acid fragments of interest, producing “adaptored fragments.” In this approach in vitro transcription from the adaptored fragments generates RNA products (“signature transcripts” or a “signature transcript mixture”) which are analyzed, e.g., using mass spectrometry, and detection of certain RNAs is evidence of the presence of specific nucleic acid sequences in the sample. See FIG. 1B and § 5.2, below.

In a third approach of the present invention, an oligonucleotide adaptor linked to chemical moiety, sometimes called a “flight moiety,” that is highly amenable to flight and detection in a mass spectrometer is ligated to the ligatable ends. See FIG. 1C and § 5.3 below.

### 2. Sequences of Interest (Target Sequences)

The invention provides rapid and economical methods for analysis or detection of polynucleotides comprising sequence(s) of interest (sometimes referred to as “target sequence(s)”) in a heterogeneous mixture of polynucleotides, most of which do not contain the target sequence(s). The polynucleotides comprising a sequence(s) of interest may be called “target polynucleotides,” the polynucleotides that do not comprise a sequences of interest may be called “background polynucleotides,” and a composition comprising target polynucleotides and background polynucleotides may be called a “complex mixture” of polynucleotides.

A complex mixture of polynucleotides may contain polynucleotides from any source containing nucleic acids (herein called a “biological source”). Exemplary biological sources include patient samples, veterinary samples, environmental samples, agricultural samples, bacterial samples, and food samples. In some cases the biological source contains DNA from an animal, such as a human patient. In some cases the biological source comprises bacteria, viruses, and/or fungi, which may be pathogenic or benign. In some cases the biological source comprises a population of microbes from a microbiome. A complex mixture of polynucleotides may comprise polynucleotide(s) from a single organism (e.g., DNA from a human single patient) or from a plurality of organisms, e.g., at least two organisms or at least three organisms, at least 25 organisms, or at least 100 organisms.

When a biological source is from an animal (e.g., human) polynucleotides may be obtained from urine, blood, plasma, serum, saliva, synovial fluid, lymph, milk, mucus, CSF, cell lysates, feces, or amniotic fluid. In some embodiments, the biological source is a sample collected from a pregnant woman that comprises both maternal polynucleotides and polynucleotides from a fetus or embryo. The target sequence may be a nucleic acid sequence that is unique to a particular genetic variant (e.g., a genetic variant associated with development of a disease), a particular pathogen or a particular pathogen gene, or a sequence that identifies a rare malignant cancer cell in a patient sample in which most cells are normal.

In general, the sequence of interest is rare in the biological source, or the complex mixture derived from it. The methods described herein may be used to detect a target sequence in a background of a large excess of non-target polynucleotides. The ratio of target polynucleotides to non-target (or ‘off-target’) polynucleotides may be 1:1×10³ or lower, sometimes 1:1×10⁴ or lower, sometimes 1:1×10⁵ or lower, sometimes 1:1×10⁶ or lower, sometimes 1:1×10⁷ or lower, sometimes 1:1×10⁹ or lower, and sometimes or lower.

Polynucleotides from a biological source may be enriched, isolated, partially isolated, or not isolated from other components of the biological source prior to use in the assay and may be amplified or may not be amplified prior to use in the assay. The polynucleotides from the biological source may be fragments of larger nucleic acids. Fragments may be produced by random fragmentation (including fragmentation during collection, purification or processing of the biological source or nucleic acids from the biological source). Alternatively, known methods may be used to produce fragments with desired characteristics (i.e., nonrandomly fragmented) before being assayed using the methods of the invention. In some approaches, polynucleotides from a biological source are processed (e.g., amplified) to produce fragments or derivatives (e.g., amplicons) for analysis. For example, RNAs from a biological source may be reverse transcribed to produce cDNA (derivatives) for analysis. In some cases, the source DNA may be used to prepare a recombinant library (derivatives) that is used on the method discussed herein. In some approaches, derivatives (e.g., amplicons or cDNAs) may be enriched for target sequences by targeted amplification or replication. Target polynucleotides may be DNA (e.g., genomic DNA, complementary DNA, mitochondrial DNA), RNA including messenger RNA, RNA or DNA from a nucleic acid library, or the like. Target polynucleotides may be single stranded or double stranded.

### 3. End-Blocked Polynucleotides

As noted above, according to the assay, polynucleotides in the complex mixture are end-blocked to render them unsuitable as substrates for ligase. That is, target and non-target polynucleotides from the complex mixture are modified so they do not have “ligatable ends” and are not potential substrates for a ligase. It will be recognized that, in the context of a particular assay, whether or not target and non-target polynucleotides is or is not a substrate for a ligase may depend to some extent on the properties of the ligase. For example, double stranded DNAs with blunt ends may not be substrates for a ligase that requires a single stranded overhang structure, but may be a suitable substrate for a blunt-end ligase. Exemplary DNA ligases used in the present method include, without limitation, T3 DNA ligase, T4 DNA ligase, T7 DNA ligase, E. coli DNA ligase, and Taq DNA ligase.

“Ligation” refers to the formation of phosphodiester bonds between the 3′-hydroxyl end of a polynucleotide with the 5′-phosphoryl end of the same or another polynucleotide. Thus, a double stranded DNA with a 5′ hydroxyl and a 3′ hydroxyl does not have a ligatable end and is end-blocked. A population of double-stranded polynucleotides with ligatable ends may be converted to a population of double-stranded polynucleotides that do not have ligatable ends, i.e., are “end-blocked,” by various art-known means. Because enzymatic ligation with most conventional ligases (e.g., T3 DNA ligase, T4 DNA ligase, T7 DNA ligase, E. coli DNA ligase, or Taq DNA ligase) requires a 5′ terminal phosphate, a polynucleotide can be “end blocked” or converted to a polynucleotide with no ligatable end (or “made unligatable”) by enzymatically or chemically removing the 5′ phosphate. Exemplary phosphatases that may be used to remove 5′ phosphate groups include calf intestinal phosphatase (CIP), shrimp alkaline phosphatase (SAP), placental alkaline phosphatase (PLAP) or secreted embryonic alkaline phosphatase (SEAP).

Alternative methods for end-blocking polynucleotides may be used, including ligation of a hairpin adaptor, ligation of an adaptor containing a chemical blocking group, ligation of an adaptor lacking a 5′ phosphate, chemical addition of a blocking group, enzyme-mediated addition of a modified nucleotide, enzyme-mediated addition of one or more nucleotides producing a sticky end overhang that is incompatible with the future ligation of the a specific adaptor, or any other method that prevents efficient downstream ligation of a functional adaptor. Still other methods for end-blocking polynucleotides, such as those described in Ukai et al., 2002, “A new technique to prevent self-ligation of DNA,” J. Biotechnol., 97:233-42, which is incorporated herein by reference, may also be used in the methods disclosed herein. Typically the end-blocked polynucleotides are double-stranded DNA.

Typically the end-blocked polynucleotides are DNA, generally double-stranded DNA, and are modified at the 3′ termini of both strands. In some embodiments, substantially all of the DNA polynucleotides in the complex mixture, or composition, are end-blocked. For example, at least about 90% of the DNA polynucleotides in the composition may be end-blocked and have no ligatable end, sometimes at least 95%, and sometimes at least about 98%.

### 4. Cleaving End-Blocked Polynucleotides with a Nucleic Acid-Directed Endonuclease(s) that Targets a Sequence(s) of Interest

The end-blocked polynucleotide composition is treated with a sequence-specific endonuclease that cleaves end-blocked DNA fragments to produce two or more subfragments, at least one of which has at least one ligatable end. Typically, the sequence-specific endonuclease is a nucleic acid-directed endonuclease that recognizes a specific target sequence in a polynucleotide, and cleaves at or near the target sequence to produce a ligatable end. In one approach, two nucleic acid-directed endonucleases with different sequence specificities cleave the same target polynucleotide to produce one or multiple fragments with two ligatable ends. A given gene or target sequence may be cut by the endonuclease or endonucleases into several pieces that, using the techniques described below, may produce a defined combination of transcripts, polypeptides, or labeled DNA fragments that in combination register a mass profile indicating that the target sequence is present in the biological source.

It will be appreciated that cleavage of polynucleotides in a complex mixture of end-blocked polynucleotide with a nucleic acid-directed endonuclease produces a composition that contains end-blocked non-target polynucleotide(s) and target polynucleotides that contain a target sequence of interest (or a sequence linked to the target sequence) and at least one ligatable end.

### 4.1 Nucleic-Acid Directed Endonuclease

In one approach, at least some of the end-blocked polynucleotides of the polynucleotide composition are digested with a nucleic-acid directed endonuclease (also called a “programmable endonuclease”) to produce fragments with ligatable ends. Depending on the programmable endonuclease used, the ligatable end produced may be blunt (as with Cas9 protein) or may have a defined overhang.

In some embodiments, the programmable endonuclease is a CRISPR-associated (Cas) protein. Examples of suitable Cas proteins, for illustration and not limitation, include Streptococcus pyogenes Cas9 nuclease, Cas9 nickase, Cas12 (Cpf1), PfAgo (Argonaute), and homologs, paralogs, orthologs, derivatives and variants of each. Variants useful in the present assay may have all of, or fewer than all of, the activities of the wild type enzyme, provided the endonuclease is able to clear a target polynucleotide in a site-specific manner. For example, a variant Cas9 protein may have all or some of the activities of Streptococcus pyogenes Cas9 as described in Woo Cho et al., Nat. Biotech, 31:230-232 (2013); Anders et al., Nat., 513:569-573 (September 2014); and Mali et al., Nat. Methods, 10:957-963 (2013). Other suitable Cas proteins are described in the scientific literature. See Makarova, K. and Koonin, E., 2015, “Annotation and Classification of CRISPR-Cas Systems,” Methods in Mol Biol 1311:47-75, incorporated herein by reference.

In some embodiments, a sequence-specific nuclease other than CRISPR associated protein or other than a programmable endonuclease (as defined above) may be used. Examples of such nucleases include TALENS and Zinc Finger Nucleases, meganucleases and other highly sequence-specific nucleases. For illustration and not limitation, programmable endonucleases that may be used in the disclosed methods are described in Zetsche et al., Cell. 163(3):759-71 (2015); Enghiad and Zhao, ACS Synth. Biol. 65:752-757 (2015); Kim et al., Nat, Rev. Genet. 15:321-34 (2014); and Guha et al., Int. J. Mol. Sci. 18:2565 (2017), each of which is incorporated herein by reference for all purposes.

### 4.2 Sequence Specificity of RNA-Directed Endonucleases (Guide RNAs)

As noted, the nucleic-acid directed endonuclease (or “programmable endonuclease”) targets a sequence of interest present in the end-blocked polynucleotide composition. The programmable endonuclease may target the sequence of interest with a “guide RNA.” The guide RNA(s) include an RNA sequence complementary to a DNA region of interest and directs the endonuclease to predefined cleavage sites in a target polynucleotides. For Cas9, the guide RNAs may be composed of two molecules, i.e., one RNA (the “crRNA”) which hybridizes to a target and provides sequence specificity, and one RNA (the “tracrRNA”), which is capable of hybridizing to the crRNA. Alternatively, the guide RNA may be a single molecule (i.e., a sgRNA) that contains crRNA and tracrRNA sequences.

The guide RNAs used in the method may be designed so that they direct binding of the endonuclease. In certain cases, the cleavage sites may be chosen so as to release a fragment that contains a sequence that identifies a gene, mutation, organism, or other information about the target polynucleotides in the complex mixture. In one approach, the cleavage sites are chosen so to release a fragment with a length in the range of 100 basepairs to 1000 base pairs. Genomic sequences of many organisms (including bacteria, fungi, plants and animals) are known, and designing guide RNAs suitable for assays described herein can be carried out using art known methods. For example, Cas9-gRNA complexes can be programmed to bind to any sequence, provided that the sequence has a PAM motif. In some embodiments, the sgRNA or crRNA can be a degenerate sequence to target relatively conserved regions.

According to the present invention, the guide RNA library is designed to create fragments with unique masses which together constitute a mass profile identifying that the target sequence. Optionally multiple different mass profiles for multiple different target sequences are produced.

The products of endonuclease cleavage will include a sequence of nucleotides with 1 to 6 or more open reading frames of varying length. Because programmable endonucleases, such as the CRISPR-Cas system, cleave DNA at precise and predictable sites and because the target sequences, or sequences near the targets are known, the sequence of fragments generated in this system can be predicted and designed as required for the particular assay. For example, given a particular target sequence of interest, fragments with ligatable ends can be selected for desirable properties including nucleotide sequence, nucleotide composition (e.g., GC content), fragment length, secondary structure of RNA transcripts encoded by the fragment, amino acid sequence, length, and tertiary structure of polypeptides encoded by the fragment, and the like. The nucleotide composition of RNAs encoded by the fragments, or amino acid composition of polypeptides encoded by the fragments can be controlled as desired. For example, the sequences of fragments produced by a programmable endonuclease can be designed to encode RNAs or polypeptides that produce an optimal signal and unique mass pattern when analyzed by mass spectroscopy. Likewise, sequences can be designed for optimal resolution of signals associated with multiple different transcripts or polypeptides associated with a plurality of different target sequences.

### 4.3 Multiplexing

Using the methods described herein, multiple sequences of interest from a polynucleotide composition may be simultaneously analyzed by multiplexing. In this approach, the method may comprise digesting the polynucleotide composition with a plurality of defined nucleic acid-directed endonucleases. In these embodiments, the method may make use of a plurality of guide RNAs. For example, the method may make use of a set of at least 2 guide RNAs (e.g., at least 5, at least 10, at least 100, at least 1,000, or at least 10,000 different guide RNAs) that are each complementary to a different, defined, site in one or more genomes.

Methods of multiplexing and designing a plurality of guide RNAs are known in the art and are described, for example, in Jakoc̆iūnas et al., 2015, Metab. Engineering. 28:213-222; Sakuma et al., 2014, Sci. Reports, 4:5400; and Ousterout et al., 2015, Nat. Comms., 6:6244.

### 4.4 Additional Embodiments

Although this disclosure is largely presented in terms of analysis of DNA (particularly double-stranded DNA) target polynucleotides in a biological source, the target polynucleotide may be, without limitation, RNA, ssDNA or dsDNA. A person of ordinary skill in the art guided by this disclosure will be able to application the teachings herein to various forms of nucleic acid. For example, a second-strand synthesis step may be included to convert single-stranded DNA to a double-stranded molecule. As another example, an RNA can be detected by converting the RNA to double-stranded cDNA using well known methods, and conducting an analysis of that product. Alternatively, programmable endonuclease (e.g., CRISPR/Cas) that acts directly on RNA (e.g., Cas13) or on ssDNA (e.g., Cas14) may be used to produce fragments. In this approach, the RNA or ssDNA polynucleotides are end-blocked (e.g., by removing the 5′ phosphate or other methods) and an RNA ligase or an single-strand DNA ligase may be used to add RNA or DNA adaptor sequences. The sample can then be processed (e.g., amplified, transcribed, or translated as discussed above). For illustration, an adaptor containing an RNA polymerase promoter can be ligated to a ligatable end of a cleaved target RNA molecule and translation of the target RNA carried out.

### 5. Production of Analytes for Mass Spectrometric Analysis

Cleavage of target polynucleotides by the programmable endonuclease(s) produces polynucleotide fragments with ligatable ends. The ligatable ends can serve as correlates for the presence of the target sequence. The assays described herein involves using nucleic acid ligation to link a moiety to a ligatable end of a polynucleotide under conditions in which the moiety cannot be linked or ligated to a polynucleotide that lacks a ligatable end. That is to say, the combination of end blocking, substrate, and endonuclease are selected so that ligation does not occur at end-blocked termini and does occur at termini that are not blocked. The resulting moiety-target polynucleotide construct is used directly as, or indirectly to produce, an analyte that can be characterized. Generally the analyte (or more typically a combination of analytes) is characterized using mass spectrometry. For that reason, the analytes may be referred to as “mass analytes.” However, other methods of analysis may also be used.

For illustration, the following sections describe three broad classes of moieties and corresponding mass analytes.

### 5.1 Polypeptide Analytes

In this approach double-stranded or partially double-stranded oligonucleotide adaptors comprising regulatory elements for coupled in vitro transcription/translation are ligated to ligatable ends of at least some of the target polynucleotides. The addition of regulatory elements to ligatable ends of target polynucleotides results in “adaptored fragments.” An adaptored fragment may have one or two adaptors, which may be the same or different. It will be understood that the adaptor used should be compatible with the ends generated by the endonuclease. In some embodiments, the end of the adaptor that is ligated to the fragments may be blunt-ended. In other embodiments, the end of the adaptor that is ligated to the fragments may have an overhang that is complementary to the overhang generated by the endonuclease. In further embodiments, blunt-ended fragments may be A-tailed (e.g., using Taq polymerase) prior to ligation to a T-tailed adaptor. In some approaches multiple endonucleases that produce different termini (e.g., blunt, 3′ or 5′ overhangs of various lengths) are used and allow asymmetric addition of linkers. For example an endonuclease cleavage producing a 3′ overhang could be ligated to an adapter with a 5′ overhang while in the same sample, another endonuclease cleavage producing a 5′ overhang could be ligated to a different adapter with a 3′ overhang. Further multiplexing of endonucleases and adapters could be achieved by using endonucleases that generated overhangs with specific sequences, and adapters with complementary sequences.

Optionally, adaptored fragments may be amplified using the polymerase chain reaction or other well-known amplification technologies. In some embodiments, no amplification is required or carried out.

In vitro transcription from the adaptored fragments, and translation of the resulting transcripts is carried out to generate translation product(s) which may be referred to as “signature polypeptides.” The translation products are analyzed, e.g., using mass spectrometry, and detection of certain polypeptides is evidence of the presence of specific nucleic acid sequences in the sample. See FIG. 1A and § 6, below.

Preferably transcription and translation are linked or coupled. Broadly, these systems allow for the transcription of RNA from DNA and subsequent translation without the need for intervening isolation or purification of the RNA. A linked system is a two-step reaction, typically based on transcription with a bacteriophage RNA polymerase (e.g., T7 or SP6 RNA polymerase) followed by translation in rabbit reticulocyte lysate, wheat germ lysate, or E. coli cell-free systems. See Spirin et al., 1988, Science 242:1162-1164 and Shimizu et al., 2001, Nat. Biotech., 19:751-755. In a coupled system, transcription and translation occur in the same mixture. Suitable conventional systems and methods for linked or coupled transcription/translation include and systems described in, for example, Caschera et al., Biochimie, 99:162-168 (April 2014); Iskakova et al., 2006, Nucleic Acids Res., 34:e135 (October 2006); and Kim et al., Biotech. Progress, 12:645-649 (1996), and are available commercially in kit form (e.g., TnT® Quick Coupled Transcription/Translation System from Promega and PURExpress® In Vitro Protein Synthesis Kit from New England Biolabs). The adaptors may also include elements such as a start codon, transcription termination sequences, IRES element (e.g., EMCV or HCV IRES), Kozak sequence, terminator sequence, poly(A) sequence, polypeptide tag, etc. as will be well known by persons trained in the biological sciences. It will be recognized that adaptored fragments may contain two adaptors in which the upstream adaptor comprises a promoter element and the downstream adaptor provides different regulatory or stabilizing elements. The same adaptor sequence attached at different termini can serve as both a promoter and a terminator. In this approach, stop codons are introduced in the reverse frames right before the ligation site. An exemplary product produced in this approach is “[promoter]-[reverse frame stop codons]-[start codon]-[target]-[reverse-frame start codon]-[in-frame stop codons]-[reverse-frame promoter].”

As noted above, because programmable endonucleases, such as the CRISPR-Cas system, cleave DNA at precise and predictable sites, adaptors can be designed to ensure appropriate spacing of promoters and other regulatory elements relative to the fragment sequence. For example, fragments can be designed to include a suitably located start codon or to be in a desired reading frame relative to an adaptor sequence (e.g., in-frame with a start codon provided by the adaptor). In one approach cleavage sites and adaptors are selected that are intentionally nonproductive (e.g., out of frame) to simplify the number and pattern of mass analytes. For illustration, a gene of interest (e.g., an antibiotic resistance gene) may be cleaved into four different fragments with ligatable termini. It will sometimes be efficient and informative if only two of the four fragments produce polypeptide products.

### 5.2 RNA Transcript Analytes

A second approach is similar to the discussion in § 5.1, above, except that the in vitro translation component is omitted and the RNA transcript serves as the mass analyte. In this approach oligonucleotide adaptors comprising regulatory elements for in vitro transcription (e.g., promoter sequences for T3, T7 or SP6 RNA polymerases) are ligated to ligatable ends of at least some of the target polynucleotides. In vitro transcription is carried out using art known methods

As discussed in Tang et al., 1997, Anal Chem 69:331-335, incorporated herein by reference for all purposes, RNA may be suitable for analysis by MALDI-TOF mass spectrometry. In particular, RNA may be less prone than DNA to fragmentation due to the stabilizing effect of the 2′-hydroxyl group.

In some embodiments, the end-blocked polynucleotide may comprise end-blocked DNA, which is digested by the programmable nuclease to form DNA fragments with one or two ligatable ends. The DNA fragments may be transcribed to form RNA fragments. For example, an adaptor compatible with in vitro transcription may be ligated to a ligatable end of the DNA fragments. Suitable reagents for transcription may then be added to the DNA fragments to generate RNA fragments. The produced RNA fragments may then be analyzed.

RNA polymerase that recognizes the promoter (e.g., T3 RNA polymerase, T7 RNA polymerase, or SP6RNA polymerase) methods of producing RNA by in vitro transcription are disclosed in Beckert et al., Methods Mol. Biol. 703:29-41 (2011), which is incorporated by reference.

### 5.3 Association of Flight Moieties with Polynucleotides with Ligatable Ends

### 5.3.1 Adaptors Linked to Flight Moieties

In a third approach adaptors linked to mass labels are ligated to the ligatable ends of the ligase will attach a chemical moiety (referred to as a “Flight Moiety,” “Mass Tag,” or “Mass Label”) that is highly amenable to flight and detection in a mass spectrometer. As a result, the adaptored polynucleotides are associated with the mass label. Ligation of mass labels changes the mass (and optionally charge) of the adaptored polynucleotides/mass analytes and facilitates the identification of target polynucleotides relative to unlabeled non-target sequences.

A wide variety of mass labels are known in the mass spectrometry art and are suitable for tagging nucleic acids. Examples include addition of pyrylium salts (see, e.g., Waliczek et al., 2016, “Peptides Labeled with Pyridinium Salts for Sensitive Detection and Sequencing by Electrospray Tandem Mass Spectrometry,” Sci Rep 6:37720), addition of charged N-hydroxysuccinimide ester tags such as those described by Lee et al (Lee et al., 2004, “MALDI-TOF Mass Spectrometry through Charge Derivatization” Anal. Chem 76:4888-4893), addition of 4-sulfophenyl isothiocyanate (SPITC) (Leon et al., 2007, “Improved protein identification efficiency by mass spectrometry using N-terminal chemical derivatization of peptides from Angiostrongylus costaricensis, a nematode with unknown genome,” J Mass Spectrom. 42:781-92), and esterification (Lecchi et al., 2005, The Role of Esterification on Detection of Protonated and Deprotonated Peptide Ions in Matrix Assisted Laser Desorption/Ionization (MALDI) Mass Spectrometry (MS),” J Am Soc Mass Spectro. 16:1269-1274.

The mass of one base pair is approximately 650 daltons and the mass difference between a GC base pair and an AT base pair is about 1 dalton, allowing determination of the mass and GC content of any given fragment and providing the opportunity to distinguish single and double-stranded nucleic acid (e.g., DNA) fragments based on length and nucleotide composition.

### 5.3.2 Primers Associated with Mass Labels

In a related approach, an adaptor is ligated to polypeptides with ligatable ends and a primer directed amplification (e.g., PCR) is carried out in the presence of oligonucleotide primers attached to a flight moiety and containing sequences complementary to a primer binding site on the adaptors. This results in a mass analyte associated with the mass label.

### 6. Mass Spectrometry

One analytical method for measuring the mass profile of DNA fragments, RNA fragments, and/or polypeptide mixtures is mass spectrometry. Mass spectrometry broadly refers to an analytical technique in which samples are ionized into charged molecules, the ratio of their mass-to-charge (m/z) is measured. Analytes may be characterized based on the measurements. The results of mass spectrometry analysis are typically presented as a mass spectrum, which plots intensity or prevalence as a function of mass.

Various techniques for ionizing for mass spectrometric analysis are known in the art, and any method capable of producing gas phase ions from the DNA fragments, RNA fragments, and/or polypeptide mixture may be used. In preferred embodiments, the ionization imparts minimal residual energy, so as to minimize fragmentation during analysis. Examples of suitable ionization processes include fast atom bombardment (FAB), chemical ionization (CI), atmospheric-pressure chemical ionization (APO), electrospray ionization (ESI), and matrix-assisted laser desorption/ionization (MALDI) including MALDI-TOF (MALDI combined with a time-of-flight analyzer).

Various techniques for mass selection of ions are known in the art, and any method capable of separating the mass so as to reliably determine the mass profile of tagged DNA fragments, RNA fragments, and/or polypeptides produced according to the methods described herein may be used. Examples of suitable mass analyzers include sector field mass analyzers, time-of-flight (TOF) analyzers, quadrupole mass analyzers, and cylindrical ion trap (CIT) analyzers.

In some embodiments, analysis is carried out using MALDI-TOF mass spectrometry. The use of MALDI-TOF in analyzing biological samples is well-known. See, e.g., in Tost et al., Mass Spec. Revs., 21:388-418 (2002); Ayers et al., J. Clin. Microbiol. 40(9):3455-62 (2002); and Garvin et al., Nat. Biotech., 18(1):95-7 (2000), each of which is incorporated herein by reference for all purposes.

### 7. Data Analysis

In some embodiments, the analysis comprises measuring the mass profile of the DNA fragments, the RNA fragments, and/or the polypeptide mixture. As discussed above, the end-blocked polynucleotide may be digested by a plurality of programmable endonucleases so as to create fragments of unique length and mass. In some embodiments, the plurality of programmable endonucleases are selected to produce combinations of fragments having unique masses and/or identifiable mass profiles. The ‘signature’ mass profile of any given target polynucleotide can be calculated and/or determined empirically. For example, particles with masses of a, b, c, and d may be characteristic of the absence of a particular target polynucleotide, and masses of a, b, c, d and e, or masses of a, b, c, and f, may be correlated with the presence of the target polynucleotide.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of genetic analysis, comprising:
(a) providing a complex mixture comprising a plurality of end-blocked polynucleotides;
(b) digesting at least some of the end-blocked polynucleotides with a nucleic acid-directed endonuclease that cleaves a target polynucleotide comprising a sequence of interest to produce, if the sequence of interest is present in the complex mixture, a polynucleotide that comprises:
the sequence of interest and
at least one ligatable end generated by endonuclease cleavage;
(c) ligating a moiety to a ligatable end of the polynucleotide under conditions in which the moiety cannot be ligated to a polynucleotide that lacks a ligatable end to produce a moiety-target polynucleotide construct;
(d) detecting, using mass spectrometry, the moiety-target polynucleotide construct or a transcription or translation product produced from the moiety-target polynucleotide;
(e) correlating the detection of the moiety-target polynucleotide with the presence of the sequence of interest in the complex mixture.

2. A method of genetic analysis, comprising:
(a) providing a complex mixture comprising a plurality of end-blocked polynucleotides;
(b) digesting at least some of the end-blocked polynucleotides with a nucleic acid-directed endonuclease that cleaves a target polynucleotide comprising a sequence of interest to produce, if the sequence of interest is present in the complex mixture, a polynucleotide that comprises:
the sequence of interest and
at least one ligatable end generated by endonuclease cleavage;
(c) ligating a moiety to a ligatable end of the polynucleotide under conditions in which the moiety cannot be ligated to a polynucleotide that lacks a ligatable end to produce a moiety-target polynucleotide construct, wherein the moiety is a nucleotide adaptor comprising an RNA promoter and the moiety-target polynucleotide construct is configured so that transcription initiated at the promoter transcribes at least a portion of the sequences of interest to produce RNA products;
(d) translating the RNA products to produce a polypeptide or mixture of polypeptides;
(e) detecting, using mass spectrometry, the polypeptide or mixture of polypeptides; and
(f) correlating the detection of the polypeptide or mixture of polypeptides with the presence of the sequence of interest in the complex mixture.

3. The method of claim 2 wherein the in vitro transcription and translation are coupled.

4. A method of genetic analysis, comprising:
(a) providing a complex mixture comprising a plurality of end-blocked polynucleotides;
(b) digesting at least some of the end-blocked polynucleotides with a nucleic acid-directed endonuclease that cleaves a target polynucleotide comprising a sequence of interest to produce, if the sequence of interest is present in the complex mixture, a polynucleotide that comprises:
the sequence of interest and
at least one ligatable end generated by endonuclease cleavage;
(c) ligating a moiety to a ligatable end of the polynucleotide under conditions in which the moiety cannot be ligated to a polynucleotide that lacks a ligatable end to produce a moiety-target polynucleotide construct, wherein the moiety is a nucleotide adaptor comprising an RNA promoter and the moiety-target polynucleotide construct is configured so that transcription initiated at the promoter transcribes at least a portion of the sequences of interest to produce RNA products;
(d) detecting, using mass spectrometry, the RNA products; and
(e) correlating the detection of the RNA products with the presence of the sequence of interest in the complex mixture.

5. A method of genetic analysis, comprising:
(a) providing a complex mixture comprising a plurality of end-blocked polynucleotides;
(b) digesting at least some of the end-blocked polynucleotides with a nucleic acid-directed endonuclease that cleaves a target polynucleotide comprising a sequence of interest to produce, if the sequence of interest is present in the complex mixture, a polynucleotide that comprises:
the sequence of interest and
at least one ligatable end generated by endonuclease cleavage;
(c) ligating a moiety to a ligatable end of the polynucleotide under conditions in which the moiety cannot be ligated to a polynucleotide that lacks a ligatable end to produce a moiety-target polynucleotide construct, wherein the moiety is a an adaptor linked to a mass label to produce adaptored polynucleotides linked to the mass label;
(d) detecting, using mass spectrometry, the RNA adaptored polynucleotides linked to the mass label; and
(e) correlating the detection of the adaptored polynucleotides linked to the mass label with the presence of the sequence of interest in the complex mixture.

6. The method of claim 1 in which the nucleic acid-directed endonuclease is a CRISPR-associated protein (Cas) and a guide RNA.

7. The method of claim 1 in which the sequence of interest is a gene sequence of a strain of bacteria.
